(19) United States
(12) Patent Application Publication
(10) Pub. No.: US 2017/0151449 A1
(43) Pub. Date: Jun. 1, 2017

(54) APPARATUS FOR FOCUSED ULTRASOUND OPERATION WITH CONTROLLABLE ROTATION ANGLE OF CARTRIDGE, FOCUSED ULTRASOUND OPERATION SYSTEM, AND METHOD USING THE SAME

(71) Applicant: HIRONIC CO., LTD., SEONGNAM-SI (KR)

(72) Inventors: YOUNG-CHUN RO, ANSAN-SI (KR); SANG-HYEON HWANG, SEOUL (KR); DONG-IK KIM, SEOUL (KR)

(73) Assignee: HIRONIC CO., LTD., SEONGNAM-SI (KR)

(21) Appl. No.: 15/358,896

(22) Filed: Nov. 22, 2016

(30) Foreign Application Priority Data
Nov. 27, 2015 (KR) 10-2015-0167928

(51) Int. Cl. A61N 7/02

(52) U.S. Cl. CPC A61N 7/02; A61N 2007/0091

(57) ABSTRACT

The present disclosure relates a focused ultrasound operation apparatus suitable for providing a controllable rotation angle. The focused ultrasound operation apparatus includes: a handpiece provided for manipulation by an operator; a cartridge of a body-insertion type which is detachably coupled to the handpiece and includes an ultrasound emitting portion. In particular, the handpiece includes: a rotation module portion provided for the operator to rotate the cartridge coupled to the handpiece; a case arranged outside the rotation module portion; and a cartridge rotation angle adjustor to adjust a rotation angle of the cartridge with respect to a rotation motion around a center axis in a body-insertion direction of the cartridge, interacting with a rotation of the rotation module portion.

### CROSS-REFERENCE TO RELATED APPLICATION

This application claims the priority to and the benefit of Korean Patent Application No. 2015-0167928, filed on Nov. 27, 2015, which is hereby incorporated by reference in its entirety.

### FIELD

The present disclosure relates to an operation apparatus for precisely controlling a cartridge during a focused ultrasound operation, a system comprising the same, and an operation method thereof.

### BACKGROUND

Ultrasound waves refer to pulses having a frequency of 20 KHz or more and have been used in various ways for skin aesthetics as well as diagnoses and treatments of lesions in the medical field. In particular, ultrasound waves in the form of being focused with high intensity are referred to as a high-intensity focused ultrasound (HIFU), and an apparatus that generates the HIFU is referred to as a high-intensity focused ultrasound generation apparatus.

A general high-intensity focused ultrasound generation apparatus includes a transducer for emitting ultrasound waves and generates heat by focusing the emitted ultrasound waves on one point (hereinafter, referred to as the “focal point”), thereby causing a rapid temperature increase in an operating region. Accordingly, intended medical operations may be performed on various lesions by using such a hyperthermia function without leaving side effects.

The skin structure of a human body may include an epidermis layer, a dermis layer, a hypodermis layer in sequence from the outer surface. A muscle layer, and a skeleton structure reside under the hypodermis layer. Among the above layers, most materials forming the dermis include collagen which gives skin elasticity.

The HIFU does not act on the epidermis layer, but acts on a superficial musculo-aponeurotic system (SMAS) layer, thereby inducing a coagulation operation and transferring heat to a deep part of the dermis via ultrasound. The HIFU is used for skin aesthetics. The HIFU may be applied to gynecologic disease patients. As described above, various gynecologic diseases may be treated or operations in the inside of a vagina may be performed by emitting focused ultrasound into the inside of a vagina.

Since it is difficult to examine the inside of a vagina with the naked eyes from the outside, it is difficult to perform a precision operation. Particularly, in order to uniformly emit the HIFU into the inside of the entire vagina, it is desired to precisely control an operation apparatus inserted into a vagina. However, we have discovered that since the inner surface of a vagina is irregular, a part may be operated twice or a part may be missed. Accordingly, precise rotation control is desired.

### SUMMARY

The present disclosure provides a focused ultrasound operation apparatus with a controllable rotation angle, by which an effect of a focused ultrasound operation is improved, the effect is maintained long, and various side effects generated after a focused ultrasound operation are reduced. In another form, the present disclosure provides an operation system including the focused ultrasound operation apparatus, and an operation method thereof.

Another aspect of the present disclosure is to provide a system and operation method for controlling a rotation angle of an operation apparatus so that a focused ultrasound operation may be precisely performed, and the operation apparatus is controlled by checking an accumulated amount of the rotation angle.

The present disclosure is not limited to the above-described effects and other advantages can be appreciated by those skilled in the art from the following descriptions.

In one aspect of the present disclosure, a focused ultrasound operation apparatus comprises: a handpiece provided for manipulation by an operator; and a cartridge of a body-insertion type detachably coupled to the handpiece and comprising an ultrasound emitting portion that is inserted into a human body and configured to generate focused ultrasound waves. In particular, the handpiece comprises: a rotation module portion provided for manipulation by the operator to rotate the cartridge coupled to the handpiece; a case arranged outside the rotation module portion; and a cartridge rotation angle adjustor configured to adjust a rotation angle of the cartridge of a body-insertion type with respect to a rotation motion around a center axis in a body-insertion direction of the cartridge, interacting with a rotation of the rotation module portion.

In another aspect of the present disclosure, a focused ultrasound operation system comprises: a focused ultrasound operation apparatus configured to generate focused ultrasound waves to form a thermal focal point by the focused ultrasound waves at a certain depth from a skin surface; a manipulation portion configured to control ultrasound emission of the focused ultrasound operation apparatus; a monitoring portion configured to monitor an operation state of the focused ultrasound operation apparatus; and a main body portion configured to control the focused ultrasound operation apparatus, the manipulation portion, and the monitoring portion. In particular, the focused ultrasound operation apparatus comprises: a handpiece provided for manipulation by an operator; and a cartridge of a body-insertion type detachably coupled to the handpiece and comprising an ultrasound emitting portion that is inserted into a human body and configured to generate focused ultrasound waves. The handpiece comprises: a rotation module portion provided for manipulation by the operator to rotate the cartridge coupled to the handpiece; a case arranged outside the rotation module portion; and a cartridge rotation angle adjustor configured to adjust a rotation angle of the cartridge of a body-insertion type with respect to a rotation motion around a center axis in a body-insertion direction of the cartridge, interacting with a rotation of the rotation module portion.

In one aspect of the present disclosure, a focused ultrasound operation method is performed by using a focused ultrasound operation system which comprises a focused ultrasound operation apparatus generating focused ultrasound waves to form a thermal focal point by the focused ultrasound waves at a certain depth from a skin surface. The focused ultrasound operation apparatus comprises: a handpiece provided for manipulation by an operator; and a cartridge of a body-insertion type detachably coupled to the handpiece and comprising an ultrasound emitting portion that is inserted into a human body and configured to generate focused ultrasound waves; a manipulation portion configured to control ultrasound emission of the focused ultrasound operation apparatus; a monitoring portion configured to monitor an operation state of the focused ultrasound operation apparatus; and a main body portion configured to control the focused ultrasound operation apparatus, the manipulation portion, and the monitoring portion. The method comprises: emitting focused ultrasound waves to a position at a certain depth from a skin surface by controlling the focused ultrasound operation apparatus; monitoring whether the cartridge of a body-insertion type is rotated with respect to the handpiece, by using the monitoring portion; and displaying information about a rotation of the cartridge of a body-insertion type measured by the monitoring portion, which is performed by the main body portion.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a perspective view illustrating an external appearance of an operation apparatus in one form of the present disclosure, in which a cartridge is controlled to rotate precisely;

FIG. 2 is an exploded view illustrating a combination relationship among constituent elements of an operation apparatus in one form of the present disclosure;

FIG. 3A is a cross-sectional view of a rotation coupling portion and a rotation adjustment portion, in one form of the present disclosure;

FIG. 3B is an enlarged view of the portion indicated in FIG. 3A;

FIG. 4 illustrates a structure of the rotation adjustment portion and the rotation coupling portion, in one form of the present disclosure;

FIG. 5 illustrates a structure of the rotation adjustment portion and the rotation coupling portion for rotating the cartridge in one direction, in another form of the present disclosure;

FIG. 6 illustrates a structure in which the rotation coupling portion has elasticity, in one form of the present disclosure;

FIG. 7 illustrates a configuration of a system with a focused ultrasound operation apparatus coupled thereto, in one form of the present disclosure;

FIG. 8 is a block diagram illustrating a process in which a handpiece of an operation apparatus transfers information about a rotation of the cartridge to the system, in one form of the present disclosure;

FIG. 9 illustrates a constituent element to detect a rotation angle, in one form of the present disclosure;

FIG. 10 illustrates a constituent element to detect a rotation angle, in one form of the present disclosure; and

FIG. 11 is a flowchart of a process of performing an ultrasound emission operation by detecting a rotation angle, in one form of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The above features and advantages will become apparent from the detailed description with reference to the accompanying drawings. Forms are described in sufficient detail to enable those skilled in the art in the art to easily practice the technical idea of the present disclosure. Detailed descriptions of well known functions or configurations may be omitted in order not to unnecessarily obscure the gist of the present disclosure.

As the disclosure allows for various changes and numerous forms, forms will be illustrated in the drawings and described in detail in the written description. However, this is not intended to limit the present disclosure to particular modes of practice, and it is to be appreciated that all changes, equivalents, and substitutes that do not depart from the spirit and technical scope of the present disclosure are encompassed in the present disclosure. In the description of the present disclosure, certain detailed explanations of the related art are omitted when it is deemed that they may unnecessarily obscure the essence of the disclosure.

Terms such as “first,” “second,” “A,” “B,” “(a)” and “(b)” are used herein merely to describe a variety of constituent elements, but the constituent elements are not limited by the terms. Such terms are used only for the purpose of distinguishing one constituent element from another constituent element. When a constituent element “connects” or is “connected” to another constituent element, the constituent element may be construed to be directly connected to the other constituent element or to be connected to the other constituent element through at least one of other constituent elements.

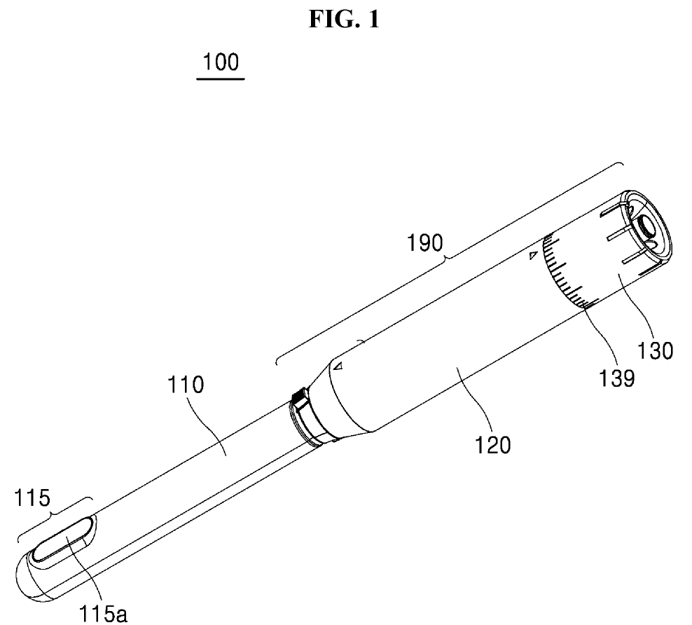

FIG. 1 is a perspective view illustrating an external appearance of an operation apparatus 100 in one form of the present disclosure, in which a cartridge 110 is controlled to rotate precisely. Detailed constituent elements of the operation apparatus 100 are described as follows.

Referring to FIG. 1, the cartridge 110 may include an ultrasound emitting portion 115 that emits a high-intensity focused ultrasound (HIFU). In detail, the cartridge 110 has a shape to be inserted into the inside of a vagina of one who will receive an operation (e.g., patient). For example, the cartridge 110 may have a shape of a circular bar or a regular polygon. A tip end of a regular polygon is processed to be round so that pain of the patient may be reduced. To reduce the patient's pain, the regular polygon may be a regular decagon, but not limited thereto.

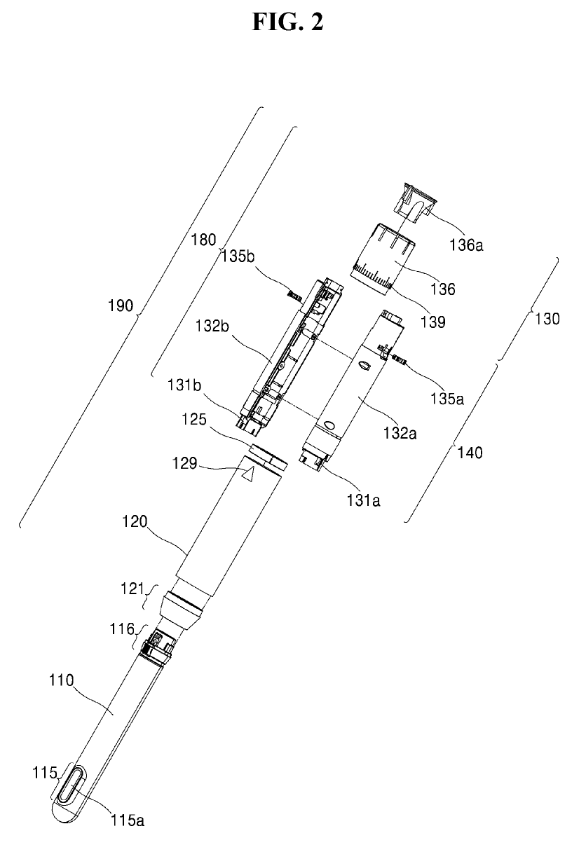

Furthermore, according to the present form, the cartridge 110 to be inserted has a circular section, and a case 120 and a rotation module portion 180 (see FIG. 2) of a handpiece 190 (see FIG. 2) may have a regular polygonal section. In this case, an operator may effectively recognize a degree of rotation of the cartridge 110. For example, when the case 120 and the rotation module portion 180 have a regular dodecagonal section, it may be seen that a degree of rotation of one side of the polygon is approximately 30°, which may facilitate an operation by the operator.

Although it is not illustrated, the ultrasound emitting portion 115 may emit focused ultrasound waves to an operating region by using a constituent element that is a so-called transducer. The transducer is arranged in the cartridge 110. In the present form, the transducer may emit the HIFU. The transducer may emit ultrasound waves in a fixed state or in a back-and-forth linear movement or a left-and-right rotation movement. The ultrasound emitting portion 115 may include one or more transducers. In one form, the transducers may be arranged so that the HIFU may be emitted in two or more different directions. The ultrasound emitting portion 115 may include a window 115a through which the ultrasound waves of the transducer are transmitted to the outside.

Referring to FIG. 2, one end 116 of the cartridge 110 may be coupled to one end of a rotating body 140. A portion of a cartridge rotation manipulator 130 is connected to the rotating body 140, and the rotating body 140 may be arranged in the case 120. The rotating body 140 arranged in the case 120 may interact with a rotation of the cartridge rotation manipulator 130 and a rotation of the cartridge 110 of a body-insertion type. The rotating body 140 may be detachably coupled to the opposite end of the one end of the cartridge 110 where the ultrasound emitting portion 115 is arranged, but the present disclosure is not limited thereto.

The case 120 is located outside the rotating body 140 and rotates by a certain angle maintaining a constant gap from the rotating body 140. The case 120 may have a hollow structure.

The cartridge 110 may be provided in a variety of types and may have a variety of shapes. Even when the shape and type of the cartridge 110 are provided in various ways, the one end 116 of the cartridge 110 coupled to the rotating body 140 may be the same size. Accordingly, the operator may use the operation apparatus 100 by coupling various cartridges to the same rotating body 140 according to the patients or operation methods.

The cartridge rotation manipulator 130 and the rotating body 140 together are referred to as the rotating module portion 180.

A cartridge rotation angle adjustor adjusts a rotation angle of the cartridge 110 with respect to a rotation motion around a center axis in a body-insertion direction of the cartridge 110 of a body-insertion type in the interaction with the rotation of the cartridge rotation manipulator 130. The cartridge rotation angle adjustor may be arranged in the case 120 of the handpiece 190.

In one form, a rotation adjustment portion, which is provided according to a rotation direction of the cartridge 110 of a body-insertion type, may be arranged on any one of the rotation module portion 180 and the case 120. A rotation coupling portion, which is provided in a structure corresponding to and interlocked with the rotation adjustment portion, may be arranged on the other one of the rotation module portion 180 and the case 120. The rotation adjustment portion and the rotation coupling portion together may be referred to as the cartridge rotation angle adjustor. Exemplary forms of the rotation adjustment portion are 135a, 135b, 135c, 135d, 135e, 135h, 135g, 635, 135 in FIGS. 2 to 6 and FIG. 8. Exemplary forms of the rotation coupling portion are 125, 125h, 625 in FIGS. 2 to 6 and FIG. 8.

In one form, the rotation adjustment portion may be a leaf spring in the form of a protrusion, whereas the rotation coupling portion may be a serration interlocked with the above-described protrusion. In another form, the rotation coupling portion may be a leaf spring in a protruding form, whereas the rotation adjustment portion may be a serration interlocked with the above-described protrusion.

In other form, the rotation adjustment portion is arranged in the form of a leaf spring in the rotation module portion 180, and the rotation coupling portion is arranged as a serration fixed in the case 120. As the rotation adjustment portion and the rotation coupling portion are coupled to each other, the cartridge 110 is rotated by a certain angle so that the operator may perform an operation by precisely rotating the cartridge 110.

The handpiece 190 may include the case 120 and the rotation module portion 180. The rotation module portion 180 may include the rotating body 140 and the cartridge rotation manipulator 130. Furthermore, in one form, the cartridge rotation manipulator 130 and the rotating body 140 may be integrally formed.

FIG. 2 is an exploded view illustrating a combination relationship among constituent elements of an operation apparatus, in one form of the present disclosure.

Referring to FIGS. 1 and 2, the one end 116 of the cartridge 110 may be detachably coupled to one end (i.e., comprising 131a and 131b) of the rotating body 140. The cartridge 110 may be provided in a variety of sizes and types. The rotating body 140 may have a half parting combination structure divided into an upper end and a lower end or may be implemented in a single parting having a hollow structure.

The upper end and the lower end of the rotating body 140 may respectively include first and second connection portions 131a and 131b connected to the cartridge 110, first and second columns 132a and 132b having a semicircular section to rotate inside the case 120, and first and second rotation adjustment portions 135a and 135b controlling precise rotation of the cartridge 110 inside the case 120. The first and second rotation adjustment portions 135a and 135b may have a leaf spring shape according to a configuration, and the rotation coupling portion 125 may have a serration structure corresponding to the first and second rotation adjustment portions 135a and 135b. The cartridge rotation manipulator 130 may include first and second caps 136 and 136a.

The first and second rotation adjustment portions 135a and 135b may be coupled to the rotation coupling portion 125 arranged in the case 120, thereby controlling rotation. A connection cover portion 121 of the case 120 protects an area where the one end 116 of the cartridge 110 passes to be coupled to the rotating body 140.

In detail, the first and second columns 132a and 132b of the rotating body 140 may pass through the rotation coupling portion 125, and the first and second rotation adjustment portions 135a and 135b may be located inside the rotation coupling portion 125. The first and second rotation adjustment portions 135a and 135b and the rotation coupling portion 125 allow the cartridge 110 to rotate by a specific angle, for example, 5° or 10°, which is a certain angle obtained by dividing 360° by an equivalent interval.

When the present form is used, since the cartridge 110 is rotated by an equivalent angle, redundant irradiation of ultrasound waves onto a particular area in an ultrasound operation process may be inhibited or prevented. Furthermore, an operating area may be precisely controlled in the operation process. Marks 129 and 139 indicating degrees of rotation may be provided on the case 120 and the cartridge rotation manipulator 130, respectively, so that the operator may easily recognize a degree of rotation. In FIG. 2, the first and second rotation adjustors 135a and 135b and the rotation coupling portion 125 forming the cartridge rotation angle adjustor are provided such that the cartridge 110 is rotated while maintaining an equivalent rotation angle.

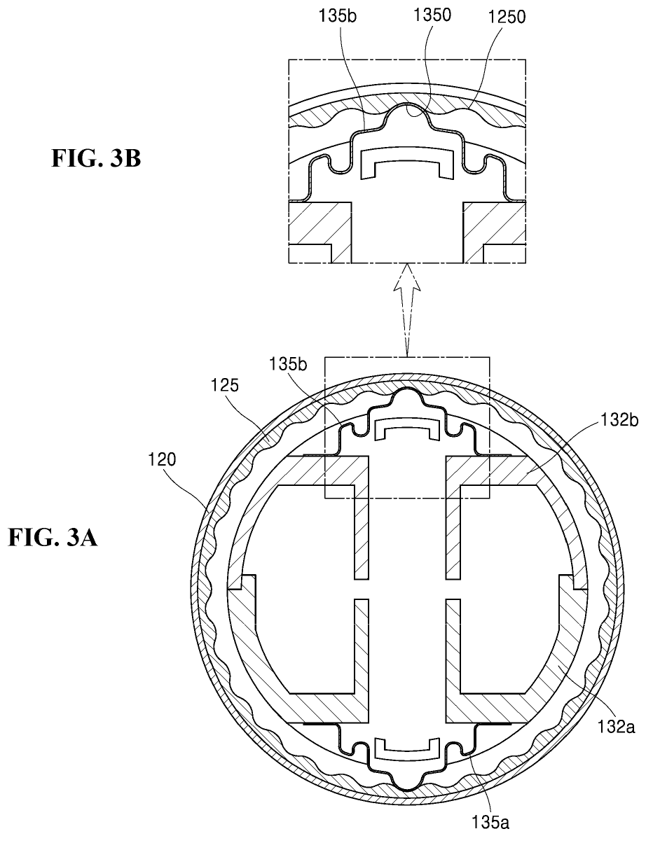

FIG. 3A is a cross-sectional view of the rotation coupling portion 125 and the first and second rotation adjustment portions 135a and 135b, in one form of the present disclosure.

Referring to FIG. 3A, the rotation coupling portion 125 may be fixedly coupled to the case 120. Although the rotation coupling portion 125 is detachable from the case 120, the rotation coupling portion 125 may be configured to rotate no longer once the rotation coupling portion 125 is located inside the case 120. In one form, the rotation coupling portion 125 may be diversely configured to control in various ways the rotation angle according to the types of cartridges. Accordingly, when the cartridge 110 is replaced, the rotation coupling portion 125 may be replaced with one of various types. For example, when the size of cartridge is large or an irradiation range of ultrasound waves irradiated is large, the rotation coupling portion 125 may be configured to have a large rotation angle.

Each of the first and second rotation adjustment portions 135a and 135b may be in the form of a leaf spring, and a protruding portion 1350 of a leaf spring may be located in a groove 1250 of the rotation coupling portion 125. Since the leaf spring has elasticity, the protruding portion 1350 moves between the grooves 1250 of the rotation coupling portion 125. However, once located in the groove 1250, the protruding portion 1350 is not easily moved. When an external rotation force is applied again, the first and second rotation adjustment portions 135a and 135b are moved to an adjacent groove 1250 of the rotation coupling portion 125 so that the cartridge 110 stops rotating after being rotated by a certain angle. The rotation angle of the cartridge 110 may vary based on the number of the grooves 1250 of the rotation coupling portion 125. For example, when the number of the grooves 1250 of the rotation coupling portion 125 is thirty six (i.e., 36), the cartridge 110 may be rotated by approximately 10° each. Likewise, when the number of the grooves 1250 of the rotation coupling portion 125 is twenty four (i.e., 24), the cartridge 110 may be rotated by approximately 15° each. As described above, the number of the grooves 1250 arranged in the rotation coupling portion 125 may be configured to be suitable or a specific cartridge.

The first and second rotation adjustment portions 135a and 135b may be arranged to face each other. Accordingly, a gap between the rotation module portion 180 and the case 120 may be maintained constant. Although in the present form the rotation adjustment portion 135 is formed of the first and second rotation adjustment portions 135a and 135b, the rotation adjustment portion 135 may be formed of three or more pieces to adjust and maintain a more precise gap.

The protruding portion 1350 of each of the first and second rotation adjustment portions 135a and 135b and the groove 1250 of the rotation coupling portion 125 may be configured in various ways, for example, a circle having a curvature or a polygon. In other words, when the rotation adjustment portion 135 is a leaf spring, as illustrated in the enlarged drawing (i.e., FIG. 3B), the rotation adjustment portion 135 may has the protruding portion 1350, and the rotation coupling portion 125 may be configured to be a serration in which the groove 1250 is formed in a shape corresponding to the protruding portion 1350.

As illustrated in FIGS. 2, 3A and 3B, the rotation module portion 180 may include the rotating body 140 arranged in the case 120 and the cartridge rotation manipulator 130 connected to the one end of the rotating body 140. The first and second rotation adjustment portions 135a and 135b and the rotation coupling portion 125 are interlocked with each other by a coupling force due to a certain elasticity. In this state, when a force greater than the coupling force is applied to the cartridge rotation manipulator 130, the cartridge 110 of a body-insertion type may be sequentially rotated by a certain rotation angle each.

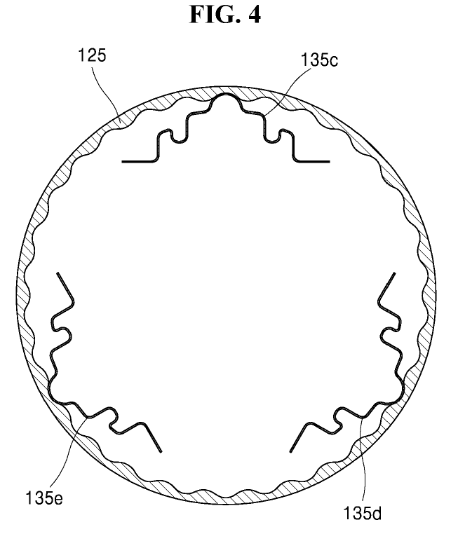

FIG. 4 illustrates a structure of the rotation adjustment portion 135 and the rotation coupling portion 125, in another form of the present disclosure. Referring to FIG. 4, for convenience of explanation, third to fifth rotation adjustment portions 135c, 135d, and 135e and the rotation coupling portion 125 only are illustrated. While in FIG. 3A the first and second rotation adjustment portions 135a and 135b are arranged spaced apart by approximately 180° from each other, the third to fifth rotation adjustment portions 135c, 135d, and 135e in the form of a leaf spring are arranged spaced apart by approximately 120° from one another. In FIGS. 3A, 3B and 4, the number of the rotation adjustment portion 135 may increase and a plurality of rotation adjustment portions may be arranged in the same interval.

The leaf spring of FIGS. 3A, 3B and 4 is an example of the rotation adjustment portion 135, and the rotation adjustment portion 135 may be configured in various ways. For example, the rotation adjustment portion 135 may be an elastic protruding portion and the rotation coupling portion has a groove corresponding to the shape of the protruding portion.

When the structure of FIGS. 3A, 3B and 4 is used, a rotation interval or a rotation angle may be controlled and the cartridge 110 may be rotated bi-directionally.

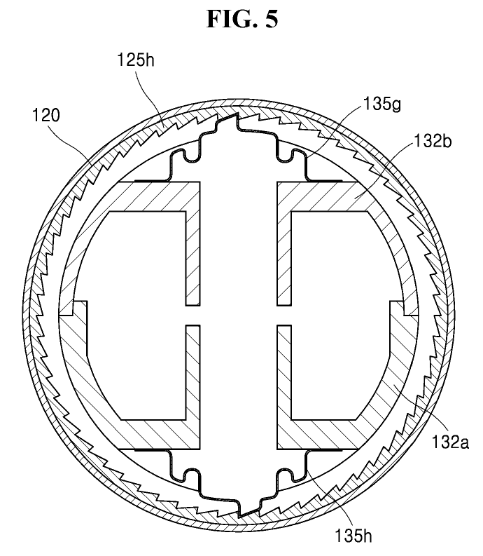

FIG. 5 illustrates a structure of the rotation adjustment portion 135 and the rotation coupling portion 125 for rotating the cartridge 110 in one direction, according to another form of the present disclosure. In other words, FIG. 5 illustrates a structure of rotating the cartridge 110 in a preset direction only unlike FIGS. 3A, 3B and 4.

Referring to FIG. 5, a protruding portion of each of rotation adjustment portions 135g and 135h is formed in an asymmetric shape. The rotation adjustment portions 135g and 135h and a rotation coupling portion 125h are coupled in a ratchet method so that the cartridge 110 may be rotated in a specific direction only. As a result, the rotation module portion 180 may be rotated counterclockwise, and the case 120 may be rotated clockwise.

FIGS. 2 to 5 illustrate a structure in which the rotation adjustment portion 135 has elasticity and the rotation coupling portion 125 does not have elasticity. In the following description, a structure in which the rotation adjustment portion 135 does not have elasticity and the rotation coupling portion 125 has elasticity is described.

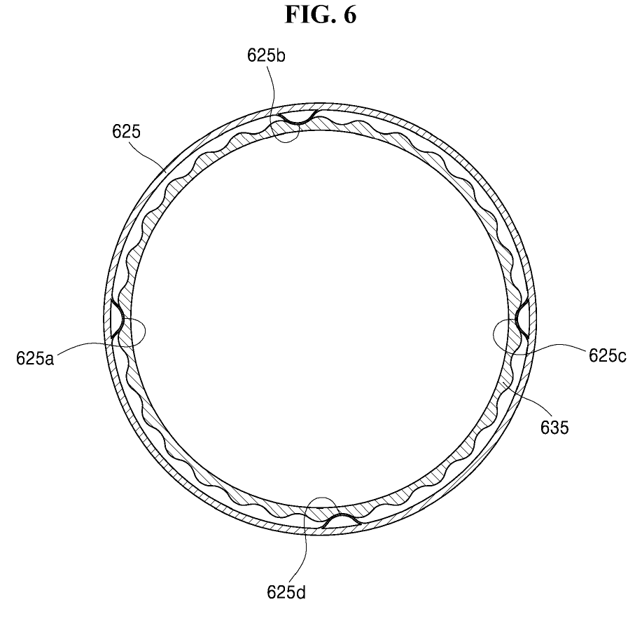

FIG. 6 illustrates a structure in which a rotation coupling portion 625 has elasticity, according to one form of the present disclosure. A plurality of elastic protruding portions 625a, 625b, 625c, and 625d may be arranged on the rotation coupling portion 625. The elastic protruding portions 625a, 625b, 625c, and 625d in a protruding form are interlocked with grooves of a rotation adjustment portion 635. The rotation adjustment portion 635, the rotation module portion 180 including the rotation adjustment portion 635, and the cartridge 110 connected to the one end of the rotation module portion 180 may be configured to be rotated by a certain angle.

In summary, as illustrated in FIGS. 2 to 6, the rotation adjustment portion and the rotation coupling portion are interlocked with each other to control the rotation of the cartridge 110. After the cartridge 110 is rotated by a certain angle, the rotation adjustment portion or the rotation coupling portion is elastically returned to the original state so that the operator may manually check the rotation angle. Also, the operator may precisely manipulate the rotation of the cartridge 110.

In the above-described forms, the cartridge and the handpiece are circular. However, the present disclosure is not limited thereto and each of any one of the cartridge and handpiece may have a regular polygonal outer shape. A tip and of a regular polygon may be formed in a gentle curve to inhibit or prevent pain to the patient. In particular, the cartridge 110 may be formed in a shape and of a material so as to be easily inserted into the body of the patient. To inhibit or prevent pain to the patient, the ultrasound emitting portion 115 is integrally formed with the cartridge 110 and thus the patient does not separately feel the existence of the ultrasound emitting portion 115 in the process of inserting the cartridge 110 into the body of the patient.

The operation apparatus 100 described with reference to FIGS. 2 to 6 is summarized as follows. The operation apparatus may include: the handpiece 190 provided for the manipulation of the operator during an operation, the ultrasound emitting portion 115 generating focused ultrasound waves, and the cartridge 110 of a body-insertion type detachably coupled to the handpiece 190. The handpiece 190 may include, with the cartridge 110 of a body-insertion type coupled to the handpiece 190, the rotation module portion 180 provided for the manipulation of the operator to rotate the cartridge 110 of a body-insertion type, the case 120 having a hollow structure and arranged outside the rotation module portion 180, and the cartridge rotation angle adjustor adjusting the rotation angle of the cartridge 110 with respect to the rotation motion around the center axis in the body-insertion direction of the cartridge 110 of a body-insertion type in the interaction with the rotation of the rotation module portion 180.

The cartridge rotation angle adjustor may include the rotation adjustment portion provided in any one of the rotation module portion 180 and the case 120 based on the rotation direction of the cartridge 110 of a body-insertion type, and the rotation coupling portion provided in the other one of the rotation module portion 180 and the case 120 in an interlocking structure corresponding to the rotation adjustment portion. The form in which the rotation adjustment portion 135 is a leaf spring and the rotation coupling portion is a serration is described in FIGS. 2 to 5, in which the rotation adjustment portion 135 is provided in a plural number. The form in which the rotation adjustment portion 635 is a serration and the rotation coupling portion 625 is a leaf spring is described in FIG. 6, in which the rotation coupling portion 625 is provided in a plural number.

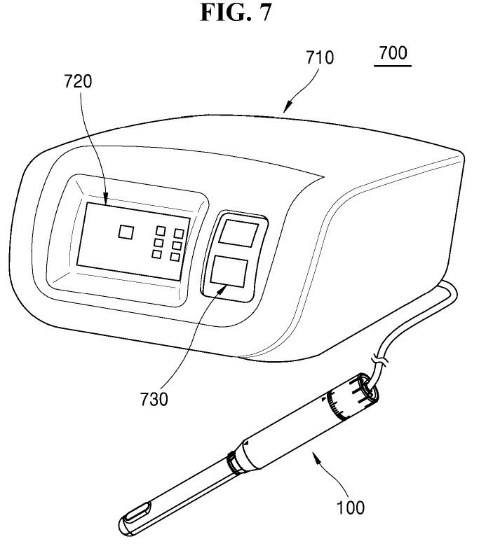

FIG. 7 illustrates a configuration of a system 700 with a focused ultrasound operation apparatus coupled thereto, according to one form of the present disclosure.

Referring to FIG. 7, the system 700 according to the present form may include: the operation apparatus 100 including the above-described cartridge 110 and handpiece 190, and a main body portion 710. The main body portion 710 may include a monitoring portion 720 for checking an operation state and a manipulation portion 730 for controlling the operation apparatus 100. The monitoring portion 720 may display an operating region that is checked by a camera in the operation apparatus 100, a current intensity of ultrasound waves emitted by the operation apparatus 100, or an elapsed operating time. In addition, the monitoring portion 720 may display various pieces of information relative to the operation of the operation apparatus 100.

The monitoring portion 720 may count a rotation angle of the cartridge 110 in the operation apparatus 100 and display the rotation angle. In one form, when the operator inserts the operation apparatus 100 into vagina of the patient and rotates the cartridge rotation manipulator 130 of the rotation module portion 180 while holding the case 120 with the operator's hand, the cartridge 110 may be rotated according to the rotation of the cartridge rotation manipulator 130. The amount of the rotation angle of the cartridge 110 may be checked from the monitoring portion 720. The checking of the amount of the rotation angle is described below.

The manipulation portion 730 of FIG. 7 controls ultrasound waves to be emitted from the ultrasound emitting portion 115 or enables manipulation of controlling a duration of emission, intensity of ultrasound waves, or a distance from an emission target position. In particular, the manipulation portion 730, in one form, may be configured to be a touch screen type. In another form, the monitoring portion 720 may be formed of a touch panel so that the manipulation control may be partially performed in the monitoring portion 720. Different menus according to the process of an operation may be displayed on a touch screen and the operator touches or drags an interface such as a button displayed on the manipulation portion 730, thereby performing the operation.

The cartridge 110 may be configured to have various diameters according to the size of the vagina of the patient. Since the cartridge 110 is detachable form the handpiece 190, the operator may replace the cartridge 110 as desired, sterilize a replaced cartridge after a one-time use for sterilization, and perform an operation using another cartridge for other patient.

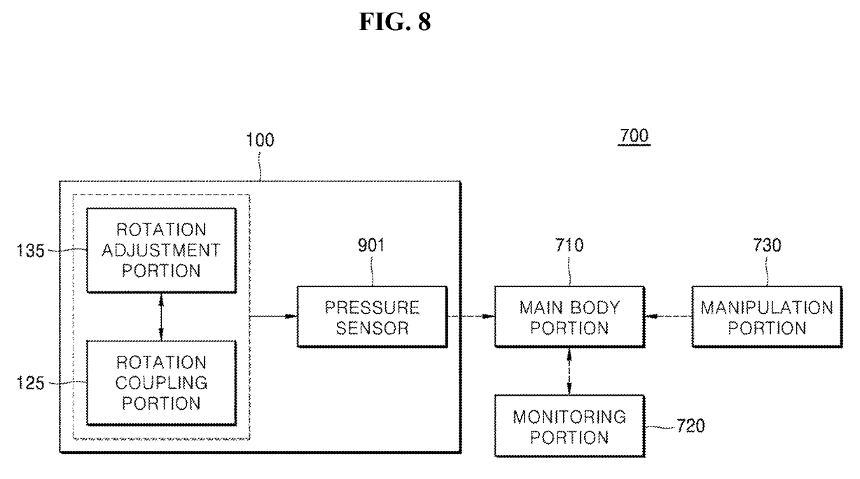

FIG. 8 is a block diagram illustrating a process in which the handpiece 190 of the operation apparatus 100 transfers information about a rotation of the cartridge 110 to the system 700, according to one form of the present disclosure. For convenience of explanation, constituent elements are indicated in rectangles and the constituent elements are applied to various forms.

In FIG. 8, the main body portion 710 of the system 700 may accumulate and store rotations between the rotation adjustment portion 135 and the rotation coupling portion 125. For example, as in the form of FIGS. 3A, 3B and 4, when the first and second rotation adjustment portions 135a and 135b are leaf springs, the number of the leaf springs being pressed by the rotation coupling portion 125 may be stored in the main body portion 710. Accordingly, the main body portion 710 may monitor the rotation angle by adding a pressure sensor at a rear end portion of the leaf spring. Alternatively, the main body portion 710 may monitor the rotation angle by adding a pressure sensor to or combining various examples of a sensor for sensing elasticity of the leaf spring with the rotation coupling portion 125. Since a rotation angle when the rotation adjustment portion 135 is pressed once may be previously determined, the number of presses of the rotation adjustment portion 135 may be recorded by using the information.

The main body portion 710 of the system 700 may control the monitoring portion 720 to display the accumulated rotation angle by using the recorded number. The operator may check the current operation state by referring to the accumulated rotation angle displayed on the monitoring portion 720.

When the accumulated rotation angle is over a preset angle, for example, over 360°, the main body portion 710 may emit ultrasound waves to the position where the first operation is performed. In order to inhibit or prevent ultrasound waves from being sequentially emitted onto an operated position, the main body portion 710 may control the operation apparatus 100 to stop the emission of ultrasound waves. In another form, the main body portion 710 may output on the monitoring portion 720 a warning screen indicating that the cartridge 110 is rotated to reach the first operating position or rotated close to the first operating position. In addition, the operator may be notified of the current operation state through warning voice or alarm. In another form, when the accumulated rotation angle is over a preset angle, an ultrasound emission condition may be changed.

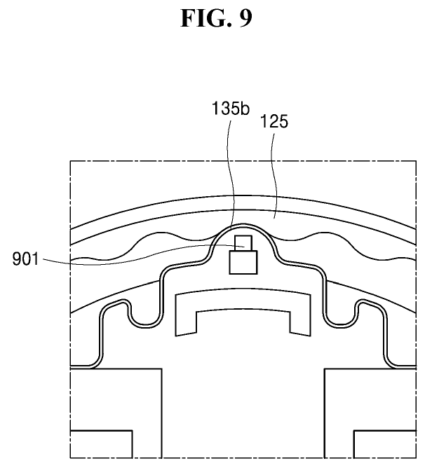

FIG. 9 illustrates a constituent element to detect a rotation angle, in one form of the present disclosure.

A pressure sensor 901 is arranged at the opposite side of the rotation coupling portion 125 with respect to the second rotation adjustment portion 135b. During the rotation process, the second rotation adjustment portion 135b may be temporarily pressed by the rotation coupling portion 125 and thus the pressure sensor 901 may be pressed by the second rotation adjustment portion 135b. The pressure sensor 901 may provide information that the pressure sensor 901 is pressed to the main body portion 710 and the main body portion 710 may store a rotation angle accumulated from a certain time point. The certain time point may be a time point when ultrasound waves are newly emitted at a specific position after the cartridge 110 is inserted into the body of the patient and moves back and forth therein. In another form, the main body portion 710 may store the rotation angle by accumulating the rotations generated in a certain area not to have an ultrasound emission area overlap each other, according to an ultrasound emission area.

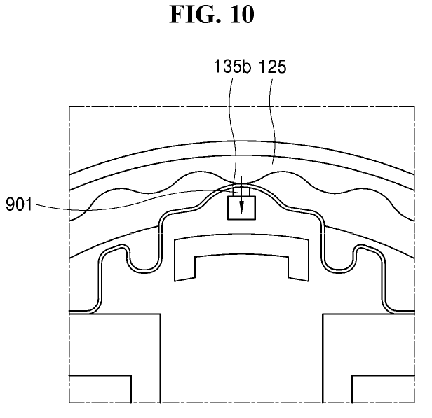

FIG. 10 illustrates a constituent element to detect a rotation angle, according to one form of the present disclosure. FIG. 10 illustrates a state in which the pressure sensor 901 of FIG. 9 is pressed by the second rotation adjustment portion 135b. The second rotation adjustment portion 135b contacts a protruding portion of the rotation coupling portion 125 and thus the second rotation adjustment portion 135b is bent and the pressure sensor 901 is pressed. Information that the pressure sensor 901 is pressed is transferred to the main body portion 710 as described above. Accordingly, the main body portion 710 may accumulate and store the rotation angle.

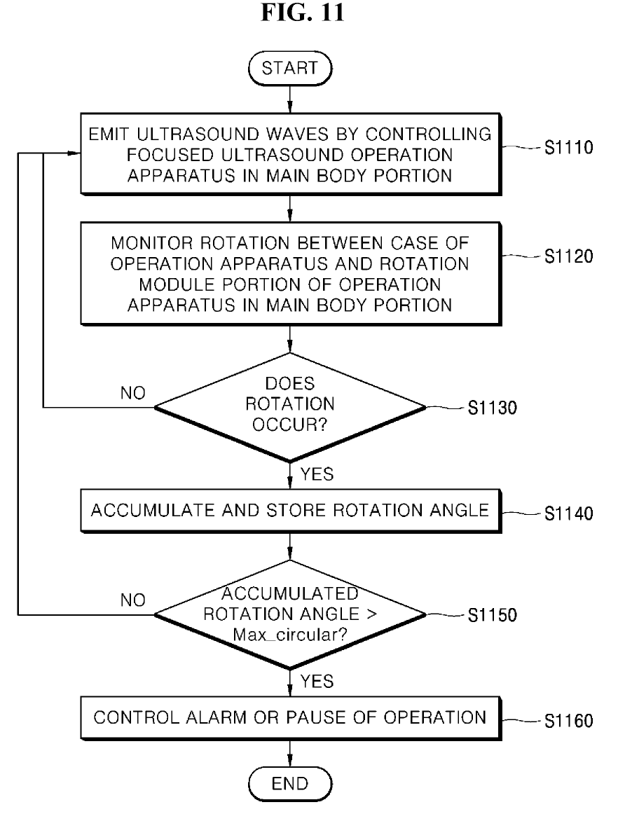

FIG. 11 is a flowchart of a process of performing an ultrasound emission operation by detecting a rotation angle, according to one form of the present disclosure. The process of FIG. 11 may be performed in the operation apparatus and the system of FIGS. 1 to 10. The process of performing an ultrasound emission operation by controlling a rotation angle performed by a high-intensity ultrasound operation system is described below.

As described above, the main body portion may emit ultrasound waves by controlling the focused ultrasound operation apparatus (S1110). In an example, the operation apparatus may be controlled by controlling the manipulation portion. Furthermore, the operation apparatus may emit ultrasound waves by a manipulation of pressing a button included in the operation apparatus.

The main body portion may monitor the rotation between the case of the operation apparatus and the rotation module portion of the operation apparatus (S1120). When the rotation occurs (S1130), the main body portion may accumulates and store the rotation angle (S1140). When the rotation does not occur, the main body portion may monitor a separate manipulation state and emit ultrasound waves.

After storing the rotation angle accumulated as the rotation occurs, the main body portion determines whether the accumulated rotation angle exceeds a preset angle Max_circular (S1150). If the accumulated rotation angle is greater than the preset angle, the main body portion may control alarming or a pause of an operation by stopping the ultrasound emission of the operation apparatus or outputting a warning screen or warning voice by controlling the monitoring portion (S1160).

When the present form is used, an operation of a gynecologic disease may be safely and precisely performed. Since the cartridge inserted into the body of the patient may be rotated by a precise angle, ultrasound waves may be uniformly emitted in the body of the patient.

In addition, when the present form is used, the operator may check a precise rotation angle through the elasticity of the rotation adjustment portion generated during rotation so that the operator may feel a sense of rotation and thus emit ultrasound waves at a precise position. Since the main body portion calculated the accumulated rotation angle of the cartridge, the operator may check whether the ultrasound waves are emitted within a rotation angle range suitable for the operation.

In the focused ultrasound operation apparatus according to the above-described forms, the operation system including the focused ultrasound operation apparatus, and the operation method thereof, a rotation angle is controlled so that an effect of a focused ultrasound operation is improved, the effect is maintained long, and various side effects generated after the focused ultrasound operation are reduced.

In the system and operation method according to the above-described forms, a rotation angle of an operation apparatus is controlled so that a focused ultrasound operation may be precisely performed, and the operation apparatus is controlled by checking an accumulated amount of the rotation angle.

The purpose of the present disclosure is not limited thereto and may include all of a high-intensity ultrasound operation apparatus, in which a rotation angle is controlled, a system, and an operation method.

The present disclosure described above may be variously substituted, altered, and modified by those skilled in the art to which the present disclosure pertains without departing from the scope and sprit of the present disclosure. Therefore, the present disclosure is not limited to the above-mentioned exemplary forms and the accompanying drawings.

What is claimed is:

1. A focused ultrasound operation apparatus, comprising:
a handpiece provided for manipulation by an operator; and
a cartridge of a body-insertion type detachably coupled to the handpiece and comprising an ultrasound emitting portion that is inserted into a human body and configured to generate focused ultrasound waves,
wherein the handpiece comprises:
a rotation module portion provided for manipulation by the operator to rotate the cartridge configured to couple to the handpiece;
a case arranged outside the rotation module portion; and
a cartridge rotation angle adjustor configured to adjust a rotation angle of the cartridge with respect to a rotation motion around a center axis in a body-insertion direction of the cartridge, interacting with a rotation of the rotation module portion.

2. The focused ultrasound operation apparatus of claim 1, wherein the cartridge rotation angle adjustor comprises:
a rotation adjustment portion provided in one of the rotation module portion and the case based on a rotation direction of the cartridge; and
a rotation coupling portion provided in other one of the rotation module portion and the case, and configured to correspond to and be interlocked with the rotation adjustment portion.

3. The focused ultrasound operation apparatus of claim 1, wherein the rotating module portion comprises:
a rotating body arranged in the case; and
a cartridge rotation manipulator connected to one end of the rotating body,
wherein the rotating body is arranged in the case and configured to interlock a rotation of the cartridge rotation manipulator with a rotation of the cartridge.

4. The focused ultrasound operation apparatus of claim 1, wherein the rotation module portion is interlocked with the cartridge by a coupling force due to elasticity of the cartridge rotation angle adjustor, and when a force greater than the coupling force is applied to a cartridge rotation manipulator, the cartridge is rotated around the center axis, maintaining an equivalent rotation angle.

5. The focused ultrasound operation apparatus of claim 1, wherein the cartridge is configured to rotate bi-directionally around the center axis.

6. The focused ultrasound operation apparatus of claim 1, wherein the cartridge performs a rotation motion only in one direction.

7. The focused ultrasound operation apparatus of claim 1, wherein the cartridge rotation angle adjustor comprises:
a leaf spring provided on one of the rotation module portion and the case based on a rotation direction of the cartridge; and
a serration fixed on other one of the rotation module portion and the case, corresponding to the leaf spring.

8. The focused ultrasound operation apparatus of claim 1, wherein the cartridge rotation angle adjustor comprises:
a plurality of leaf springs provided on one of the rotation module portion and the case based on a rotation direction of the cartridge; and
at least one serration fixed on other one of the rotation module portion and the case, and configured to correspond to the plurality of leaf springs and interlock with the plurality of leaf springs based on the rotation direction of the cartridge.

9. The focused ultrasound operation apparatus of claim 1, wherein the cartridge rotation angle adjustor further comprises a pressure sensor.

10. The focused ultrasound operation apparatus of claim 1, wherein the case is used as a handle for the operator,
the rotation module portion comprises a rotating body and a cartridge rotation manipulator, and
the rotating body is inserted in the case and has one end coupled to the cartridge and another end coupled to the cartridge rotation manipulator, the rotating body configured to interlock a rotation of the cartridge rotation manipulator with a rotation of the cartridge.

11. A focused ultrasound operation system, comprising:
a focused ultrasound operation apparatus configured to generate focused ultrasound waves to form a thermal focal point by the focused ultrasound waves at a certain depth from a skin surface;
a manipulation portion configured to control ultrasound emission of the focused ultrasound operation apparatus;
a monitoring portion configured to monitor an operation state of the focused ultrasound operation apparatus; and
a main body portion configured to control the focused ultrasound operation apparatus, the manipulation portion, and the monitoring portion,
wherein the focused ultrasound operation apparatus comprises:
a handpiece provided for manipulation by an operator; and
a cartridge of a body-insertion type detachably coupled to the handpiece and comprising an ultrasound emitting portion that is inserted into a human body and configured to generate focused ultrasound waves,
wherein the handpiece comprises:
a rotation module portion provided for manipulation of the operator to rotate the cartridge configured to couple to the handpiece;
a case arranged outside the rotation module portion; and
a cartridge rotation angle adjustor configured to adjust a rotation angle of the cartridge with respect to a rotation motion around a center axis in a body-insertion direction of the cartridge, interacting with a rotation of the rotation module portion.

12. The focused ultrasound operation system of claim 11, wherein the cartridge rotation angle adjustor comprises:
a rotation adjustment portion provided in one of the rotation module portion and the case based on a rotation direction of the cartridge; and
a rotation coupling portion provided in other one of the rotation module portion and the case, and configured to correspond to and be interlocked with the rotation adjustment portion.

13. The focused ultrasound operation system of claim 11, wherein the rotation module portion is interlocked with the cartridge by a coupling force due to elasticity of the cartridge rotation angle adjustor, and when a force greater than the coupling force is applied to a cartridge rotation manipulator, the cartridge is rotated, maintaining an equivalent rotation angle.

14. The focused ultrasound operation system of claim 11, wherein the cartridge rotation angle adjustor is provided so that the cartridge is rotated, maintaining an equivalent rotation angle.

15. The focused ultrasound operation system of claim 11, wherein the monitoring portion is configured to display information of the rotation angle of the cartridge.

16. The focused ultrasound operation system of claim 11, wherein, when a rotation angle between the rotation adjustment portion and the rotation coupling portion of the operation apparatus exceeds a preset angle range, the main body portion is configured to stop ultrasound emission of the focused ultrasound operation apparatus, change an emission condition, or configured to output a warning screen or warning voice by controlling the monitoring portion.
